(19) United States
(12) Patent Application Publication
(10) Pub. No.: US 2019/0390282 A1
(43) Pub. Date: Dec. 26, 2019

(54) TARGET ENRICHMENT AND SEQUENCING OF MODIFIED NUCLEIC ACIDS FOR HUMAN CANCER DETECTION

(71) Applicant: BioChain Institute, Inc., Newark, CA (US)

(72) Inventors: Xiaoliang Han, San Francisco, CA (US); Tong Lu, Newark, CA (US); Emil Mejares, Newark, CA (US); Eric Han, Newark, CA (US); John Lee, Newark, CA (US)

(73) Assignee: Biochain Institute, Inc., Newark, CA (US)

(21) Appl. No.: 16/419,270

(22) Filed: May 22, 2019

Related U.S. Application Data
(60) Provisional application No. 62/675,139, filed on May 22, 2018.

(51) Int. Cl. C12Q 1/6886; C12Q 1/686

(52) U.S. Cl. CPC C12Q 1/6886; C12Q 2600/112; C12Q 1/686

(57) ABSTRACT

The invention relates to a method for detecting cancer or precancerous condition in a subject. It uses rhPCR based target enrichment of nucleic acid for better sensitivity and specificity for sequencing and analysis. The method provides use of cfDNA form plasma, serum or other bodily fluids of the subject.

### FIELD OF THE INVENTION

The invention relates to a method for the enrichment of nucleic acid extracted from human plasma and a method for sequencing the same thereof. The invention in particular relates to a method of enriching and sequencing of modified nucleic acid of cell free DNA (cfDNA) that can be used in various clinical diagnostic tests particularly for human cancer detection.

### BACKGROUND

DNA methylation is an epigenetic modification that is heavily involved in regulating genome expression. Two of DNA's four bases, cytosine and adenine, can be methylated. The presence and absence of methylation in certain genetic regions has prenatal diagnostic and prognostic applications. Abnormal patterns of DNA methylation in cancer cells can be used to distinguish them from normal tissue cells. Cancers cells contain altered methylation patterns that result in aberrant expression of critical genes. In cancers, loss of expression of genes occurs about 10 times more frequently by hypermethylation of promoter CpG islands than by mutations.

The presence of circulating cell-free DNA (cfDNA—can also be referred to as circulating tumor DNA and circulating nucleic acid) in human plasma was reported in 1948 by Mendel and Metals. Recently, circulating cell-free DNA (cfDNA) has been recognized as a potential biomarker of cancer progression, treatment response, and drug resistance. The cfDNA extracted from plasma or serum of cancer patients has shown characteristics typical of tumor DNA and may serve as non-invasive biomarkers for cancer detection and management (WO2013123030A2/RYAN WAYNE L). Cell-free DNA, derived from blood or some other bodily liquid, represents a more accessible material from which DNA can be obtained for PCR and/or NGS testing and profiling.

Aberrant DNA methylation occurs early in cancer and may be detected in circulating cell-free DNA (ccfDNA), thereby constituting a valuable biomarker and enabling non-invasive testing for cancer detection. There is a need for highly sensitive methods with multiplexed detection of very low levels of methylated DNA when the majority of DNA with the same sequence is unmethylated. For example, detection of multiple methylated DNA sequences in cell-free DNA isolated from serum may enable early detection of cancer.

The drawback of the prior methods for cancer detection is amplification of unmethylated DNA along with the methylated DNA which further leads to false sequencing and analysis. There remains a need for more sensitive and specific screening tools which can identify only methylated sequences and give accurate detection. Recognizing this limitation, we developed a highly sensitive diagnostic tool for the detection of human cancers, which utilizes rhPCR (Integrated DNA Technologies, Inc.) and NGS technologies.

### SUMMARY

The present invention aims to address these needs by providing a method for the enrichment of nucleic acid from human plasma and a method for sequencing the same thereof.

It is an object of the invention, to provide a method for detecting a methylated nucleic acid to identify a cancer or precancerous condition in a subject, the method comprising the steps of: a. obtaining nucleic acid from a body fluid of the subject; b. optionally, subjecting the nucleic acid to bisulfite conversion; c. enriching the nucleic acid using rhPCR; d. sequencing of the enriched nucleic acid using NGS; and e. identifying a cancer or precancerous condition in the subject based on the sequencing; wherein rhPCR increases the sensitivity and specificity for methylated nucleic acid in the NGS based sequencing analysis by selectively amplifying methylated nucleic acid and eliminating formation of primer dimers.

According to another object of the invention, the invention provides a method for detecting a methylated nucleic acid to identify a cancer or precancerous condition in a subject, the method comprising the steps of: a. obtaining nucleic acid from body fluid of the subject; b. optionally, subjecting the nucleic acid to bisulfite conversion; c. enriching the nucleic acid using rhPCR; d. sequencing of the enriched nucleic acid using qPCR or RT-PCR (Real-time PCR); and e. identifying a cancer or precancerous condition in the subject based on the sequencing; wherein rhPCR increases the sensitivity and specificity for methylated nucleic acid in the qPCR or RT-PCR (Real-time PCR) based sequencing analysis by selectively amplifying methylated nucleic acid and eliminating formation of primer dimers.

According to another object of the invention, bisulfite conversion of the nucleic acid extracted from body fluid is performed to distinguish between the methylated cytosine and unmethylated cytosine.

According to yet another object of the invention, the rhPCR enrichment method can increase the sensitivity for methylated DNA detection with Next Generation Sequencing (NGS).

According to yet another object of the invention, the rhPCR enrichment method can increase the sensitivity for methylated DNA detection with qPCR or RT-PCR (Real-Time PCR).

According to yet another object of the invention, the rhPCR method uses uniquely designed rhPrimers and thermostable RNase H2 enzyme, which eliminate or reduce non-specific interactions such as primer-dimers or misprimed PCR products. Also, rhPCR primers selectively amplify methylation alleles, therefore markedly improves the assay sensitivity and specificity.

According to yet another object of the invention, the cancer or precancerous condition can be colon cancer, liver cancer, brain cancer, uterine cancer, bladder cancer, blood cancer, lung adenocarcinomas, breast cancer, thyroid carcinoma, pancreatic cancer, papillary thyroid carcinoma, ovarian carcinoma, gastric carcinoma, malignant, mesothelioma, prostate carcinoma, neuroblastic tumors, colorectal carcinoma, spitzoid, melanoma, salivary, esophageal carcinoma, adenoid, cystic, carcinoma, multiforme, stomach cancer, kidney cancer, urethral cancer, glioblastoma, oral squamous cell, carcinoma, mastocytosis, extramammary Paget's disease, Acute Myeloid, Leukemia, cholangiocarcinomaor sarcoma.

According to yet another object of the invention, the body fluid can be one of blood, plasma, serum, urine, saliva, ascites fluid, synovial fluid, amniotic fluid, semen, cerebrospinal fluid, follicular fluid and other body fluids.

According to yet another object of the invention, the nucleic acid is a modified nucleic acid.

According to yet another object of the invention, the nucleic acid is a methylated nucleic acid.

According to yet another object of the invention, the nucleic acid is selected from DNA, RNA, cDNA, mRNA, cfDNA, ccfDNA and ctDNA.

According to yet another object of the invention, the nucleic acid is a methylated DNA.

According to yet another object of the invention, the sequencing technique can be selected from, but not limited to, qPCR, RT-PCR (Real-time PCR), Next generation sequencing (NGS) including Illumina's MiSeq, Illumina's HiSeq, Illumina's Genome Analyzer IIX, Roche's 454 pyrosequencing, Ion torrent semiconductor, Life Technologies's SOLiD4, Life Technologies's Ion Proton, Helicos Biosciences's Heliscope and Pacific Biosciences's SMRT.

Various objects, features, aspects and advantages of the present disclosure will become more apparent from the following detailed description of preferred embodiments, along with the accompanying drawing figures.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1. shows rhPCR workflow for enrichment of target nucleic acid.

FIG. 2. shows Next Generation sequencing (NGS) assay workflow for the methylation detection through multiplex rhPCR based amplicon sequencing.

### DETAILED DESCRIPTION

The following embodiments and aspects thereof are described and illustrated in conjunction with systems, compositions and methods which are meant to be exemplary and illustrative, not limiting in scope.

### Glossary of Terms and Acronyms

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

The term “cfDNA” refers to as circulating cell free DNA that is, it is not contained in a cell and may derived from blood as it circulates freely in some biological liquids.

The term “rhPCR” refers to a novel nucleic acid amplification technology that provides improved accuracy over conventional PCR.

The term “rhPCR primers (rhPrimers)” refers to unique primers that contain RNA bases and a 3′ blocking moiety, and used in conjunction with the thermostable RNase H2 enzyme to perform rhPCR.

The term “Target enrichment” refers to amplification of target DNA. For example enrichment process can increase methylated target DNA by multiple folds than unmethylated DNA.

The term “DNA Methylation” refers to a process by which methyl groups are added to the DNA molecule.

The term “Methylated targets” refers to methylated DNA which have methyl groups added to the DNA molecule.

The term “Unmethylated targets” refers to unmethylated DNA is the dna other than the methylated DNA.

The term “bisulfite conversion” refers to a technique that involves converting cytosine to uracil while leaving 5-methylcytosine (5-mC) intact.

The term “Subject” refers to an individual who has or is at risk for developing a disease, for example subject is a human having cancer.

The term “Library preparation” refers to generating a collection similarly sized DNA fragments with known adapter sequences added to the 5′ and 3′ ends for sequencing.

Alteration in patterns of DNA methylation is a hallmark of cancer. In different types of cancers, DNA methylation in the promoter region results in silencing and inactivation of certain tumor-suppressor genes. Studies have revealed that methylated DNA markers offer scientists' a viable avenue to differentiate tumor cells from normal cells and thereby greatly improve cancer diagnosis.

Over the years, DNA bisulfite conversion has been used as a platform for various different molecular approaches. The bisulfite conversion method is based on the conversion of unmethylated cytosines into uracil (without affecting methylated cytosines) that allows for the determination of DNA methylation patterns in specific DNA regions. Bisulfited DNA is amplified by PCR using gene specific PCR primers that confer sequence specificity and high sensitivity for their subsequent determination (Yannick Delpu. Et al., DNA Methylation and Cancer Diagnosis, 2013, 14, 15029-15058). The use of Methylation-sensitivity restriction enzymes (MSRE) in combination with polymerase chain reaction (PCR) along with gene-specific primers allows for the identification of exact positioning of methylated sites (e.g. Methylated cytosine) on the genomic DNA (Anatoliy A. Melnikov et al., MSRE-PCR for analysis of gene-specific DNA methylation, 2005; 33(10): e93).

Although PCR technique is well known for detection purposes in both research and diagnosis domains, however, it may have the disadvantage of forming primer dimers and/or causing mis-amplification of homologous sequences. To overcome this limitation, PCR is performed with RNase H2 enzyme wherein the enzyme is used to activate the RNase H2 specific blocked primer hybridized to the target sequence. The RNase H2 specific primer contains a single ribonucleotide residue, which becomes the cleavage site for the enzyme to activate the primer. This eliminates primer-dimer formations and also reduces mis-amplification of related sequences. Mismatches in the cleavage region decrease the chances of enzyme cleavage, thereby further increasing the specificity of the reaction (Joseph R Dobosy et al., RNase H-dependent PCR (rhPCR): improved specificity and single nucleotide polymorphism detection using blocked cleavable primers, 2011, 11:80)

Further sequencing of PCR product can provide explicit and quantitative information of the DNA methylated patterns. Approaches like Next generation sequencing, Quantitative PCR (Real Time PCR) could be good enough to quantify the methylation level for particular region of the DNA. These approaches yield high resolution results of DNA methylation profiles, allows determination of the absolute quantity of target DNA and provide the real time visualization of amplified DNA fragments.

In one embodiment, the present invention relates to a method for detecting a methylated nucleic acid to identify a cancer or precancerous condition in a subject, the method comprising the steps of:

Step a) Obtaining nucleic acid from the body fluid of the subject;
Step b) Optionally, subjecting the nucleic acid to Bisulfite conversion;
Step c) Enriching the nucleic acid using rhPCR,
Step d) Sequencing of the enriched nucleic acid using NGS; and
Step e) identifying a cancer or precancerous condition in the subject based on the sequencing; wherein rhPCR increases the sensitivity and specificity for methylated nucleic acid in the NGS based sequencing analysis by selectively amplifying methylated nucleic acid and eliminating formation of primer dimers.

Step a) of method provided in the invention comprises obtaining nucleic acid from the bodily fluid wherein, the bodily fluid can be selected from, but not limited to, blood, plasma, serum, urine, saliva, ascites fluid, synovial fluid, amniotic fluid, semen, cerebrospinal fluid, follicular fluid and other fluid used in biopsy tests. In one example, the nucleic acid are extracted from human plasma. The extraction of the nucleic acid can be done using any of the methods and kits known in the art, but not limited to, extraction with organic solvents, Phenol-chloroform extraction, SDS-based extraction with centrifugation, QIAamp Circulating nucleic acid Kit (QIAgen Kits), MagMAX Cell-Free DNA Isolation Kit (Thermo Fisher Scientific), Quick-cfDNA™ Serum & Plasma Kit (ZYMO), Chamagic cfDNA 5k Kit (Chemagen) and NucleoSpin Plasma XS (TaKaRa) or the like.

The nucleic acid in the present invention can be selected from, but not limited to DNA, RNA, cDNA, mRNA, cell free DNA (cfDNA), circulating cell free DNA (ccfDNA), circulating tumor DNA (ctDNA) and cell-free fatal DNA (cffDNA). In one example of the invention, the modified nucleic acid is methylated. In another example, modified nucleic acid is methylated cfDNA. The invention provides use of modified nucleic acid extracted from the bodily fluids for various diagnostic tests. In one example, the modified nucleic acid are used for human cancer detection. The cancer can be, but not limited to, breast, prostate, basal cell, melanoma, colorectal, lung, brain, bladder, leukemia, lymphoma, carcinoma or related to any part of the body. In one example, the cancer is colorectal cancer.

Step b) of method provided in the invention comprises optionally, subjecting the nucleic acid to bisulfite conversion. The conversion includes treatment of nucleic acid with bisulfite or sodium bisulfite, which converts cytosine into uracil by deaminating unmethylated cytosine while leaving 5-methylcytosine (5-mC) intact. This conversion process enables PCR amplification process to recognize uracils as thymines and 5-mC or 5-hmC as cytosines allowing 5mCs to be distinguished from unmethylated cytosines.

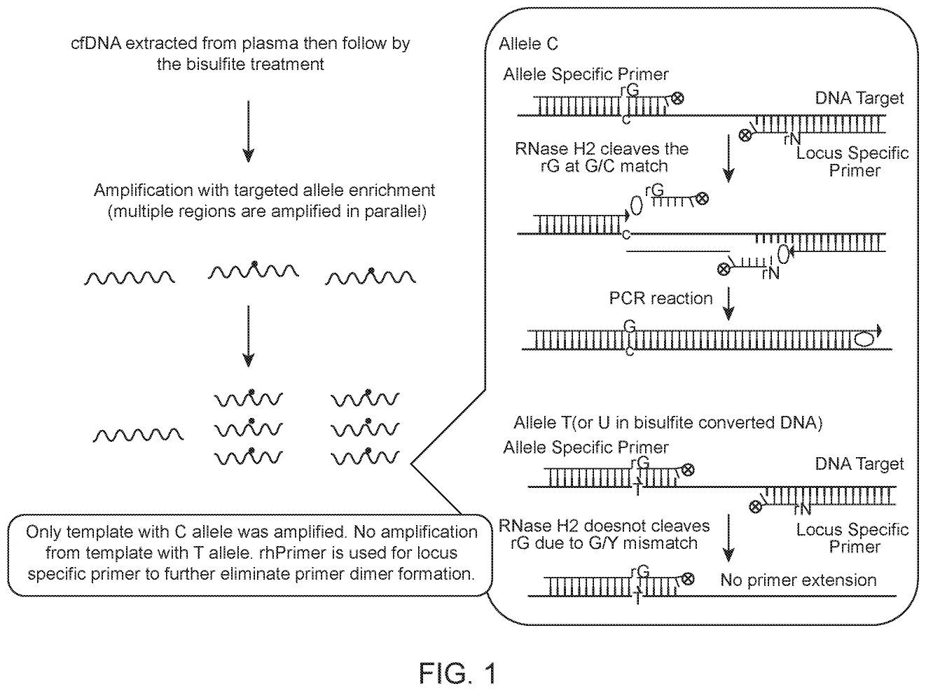

Step c) of method provided in the invention comprises enriching the nucleic acid using rhPCR as shown in FIG. 1. The rhPCR uses uniquely designed rhPrimers and thermostable RNase H2 enzyme. The enriching step comprising designing of unique rhPCR primers, wherein these rhPrimers contain a single ribonucleotide residue and a 3′ blocking moiety. The primers are blocked primers and are activated when cleaved by RNase H2 enzyme at G/C match followed by amplification step of the nucleic acid. The primers can only be cleaved after they hybridize to the perfectly matched target sequence of nucleic acid. Thus, rhPCR significantly increases the specificity and multiplexity by eliminating or reducing non-specific interactions such as primer-dimers. The rhPCR method is preceded by the bisulfite conversion facilitates the enrichment of target DNA sample further to be used for various diagnostic purposes.

The method provided in the invention comprises preparing a library of enriched nucleic acid. The library preparation involves generating a collection of nucleic acid fragments for sequencing. In one example, universal indexing PCR (24 cycles) is used for NGS DNA library preparation.

Step d) of method provided in the invention comprises sequencing of the enriched nucleic acid using NGS. The sequencing of the enriched nucleic acid enables target DNA analysis and profiling for a number of applications including, but not limiting to, diagnosing complex diseases, whole-genome sequencing, analysis of epigenetic modifications such as DNA Methylation, Gene Expression Analysis with Targeted RNA-Sequence, and mitochondrial sequencing transcriptome sequencing.

The sequencing of the enriched nucleic acid obtained from the sequencing library is achieved by Next generation sequencing (NGS) method. Various NGS methods developed by different companies can be used according to the embodiments of the invention, but not limited to Illumina (MiSeq, HiSeq, Genome Analyzer IIX), Roche (454 pyrosequencing), Ion torrent semiconductor, Life Technologies (SOLiD4, Ion Proton), Helicos Biosciences (Heliscope) and Pacific Biosciences (SMRT). In one example, the NGS is Illumina MiSeq based sequencing method wherein, Illumina Miseq includes cluster generation, amplification, sequencing, and data analysis into a single instrument.

In another embodiment of the invention, the sequencing of the enriched nucleic acid obtained from the sequencing library is achieved by Quantitative polymerase chain reaction (qPCR) or real-time PCR (RT-PCR) method.

Step e) of the method provided in the invention comprises, identifying a cancer or precancerous condition in the subject based on the sequencing. The sequenced nucleic acid are further analyzed for various diagnostic tests. In one example, the sequenced nucleic acid are analyzed for total C and T counted base on the CpG sites in the target region which indicate human cancer detection in the subject. The cancer can be, but not limited to, colon cancer, liver cancer, brain cancer, uterine cancer, bladder cancer, blood cancer, lung adenocarcinomas, breast cancer, thyroid carcinoma, pancreatic cancer, papillary thyroid carcinoma, ovarian carcinoma, gastric carcinoma, malignant, mesothelioma, prostate carcinoma, neuroblastic tumors, colorectal carcinoma, spitzoid, melanoma, salivary, esophageal carcinoma, adenoid, cystic, carcinoma, multiforme, stomach cancer, kidney cancer, urethral cancer, glioblastoma, oral squamous cell, carcinoma, mastocytosis, extramammary Paget's disease, Acute Myeloid, Leukemia, cholangiocarcinomaor sarcoma or related to any other part of the body. In one example, the cancer is colorectal cancer. In another example, the cancer is selected from Gastrointestinal Cancer, lung cancer and breast cancer.

In another embodiment, the present invention relates to a method for increasing sensitivity and specificity for methylated DNA detection using NGS based sequencing analysis and rhPCR, where rhPCR is used to enrich the methylated DNA.

In yet another embodiment, the present invention relates to a method for increasing sensitivity and specificity for methylated DNA detection using qPCR or RT-PCR (Real Time PCR) based sequencing analysis and rhPCR, wherein rhPCR is used to enrich the methylated DNA.

The method according to previous embodiments, the rhPCR is preceded by bisulfite conversion wherein, bisulfite conversion enables rhPCR to distinguish between the methylated and unmethylated cytosines.

The rhPCR primers selectively amplify methylation alleles, meanwhile eliminating the formation of primer dimers, therefore markedly improves the assay sensitivity and specificity.

The methylated DNA detection is further used to identify a cancer or precancerous condition in a subject wherein the methylated DNA is selected from, but not limited to, cDNA, cell free DNA (cfDNA), circulating cell free DNA (ccfDNA), circulating tumor DNA (ctDNA) and cell-free fatal DNA (cffDNA). In one example, the identified cancer or precancerous condition is colorectal cancer.

The rhPCR uses uniquely designed rhPrimers and thermostable RNase H2 enzyme as shown in FIG. 1. Uniquely designed rhPrimers contain a single ribonucleotide residue and a 3′ blocking moiety. The primers are blocked primers and are activated only when cleaved by RNase H2 enzyme. After removing the blocked segment from the primer, DNA polymerase is added to extend unblocked primers resulting in enriched PCR product. Thus, rhPCR significantly increases the specificity and multiplexity of the process. The rhPCR method preceded by the bisulfite conversion facilitates the enrichment of target DNA sample further to be used for various diagnostic purposes.

The rhPCR is preceded by bisulfite conversion of the nucleic acid extracted from body fluid of a subject. This conversion process enables PCR amplification process to distinguish 5mCs (methylated cytosines) from unmethylated cytosines. Compared with conventional methods, the methods of present invention can take 4-5 hours shorter.

In yet another embodiment of the invention, purification steps can be incorporated after the enrichment of nucleic acid using rhPCR method. The purification methods include, but not limited to, SPRI, DNA IQ, carboxylated beads, or the like. In one example, the nucleic acid are purified using SPRI (Solid Phase Reversible Immobilization) method.

In yet another embodiment of the invention, purification steps can be incorporated after the library preparation of enriched nucleic acid. The purification methods includes, but not limited to, SPRI, DNA IQ, carboxylated beads, or the like. In one example, the nucleic acid after the library preparation are purified using SPRI (Solid Phase Reversible Immobilisation) method.

In yet another embodiment of the invention, the enriched nucleic acid can be purified using SPRI method before and/or after nucleic acid library preparation.

In yet another embodiment of the invention, purification methods (e.g SPRI) is followed by a quantitative PCR (qPCR) to measure quantity of the nucleic acid. In one example, qPCR is performed after purification of nucleic acid libraries.

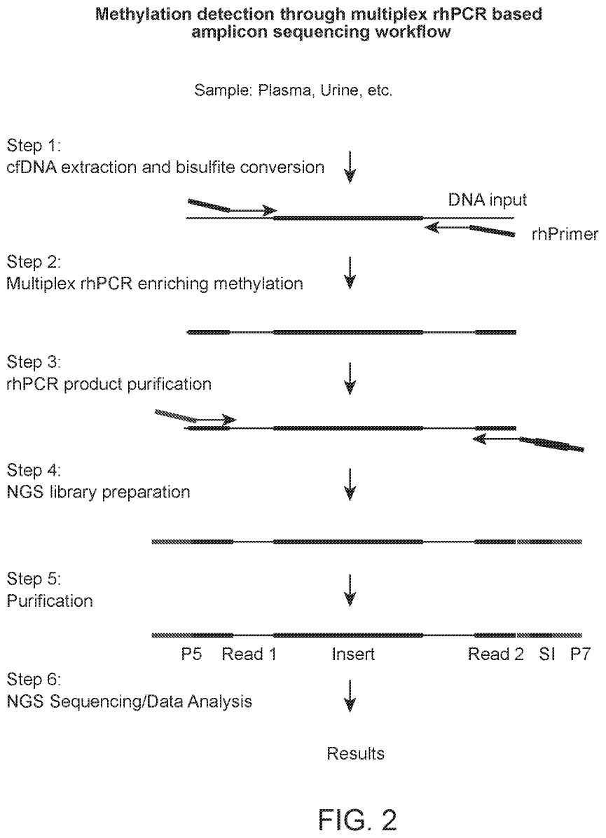

In yet another embodiment, the invention provides a method for methylation detection through multiplex rhPCR based amplicon sequencing as shown in FIG. 2. The method comprises: extracting target nucleic acid form bodily fluid sample, wherein the bodily fluid sample can be selected from but not limited to, blood, plasma, serum, urine, saliva, ascites fluid, synovial fluid, amniotic fluid, semen, cerebrospinal fluid, follicular fluid or the like; performing bisulfite conversion of the target nucleic acid to distinguish between methylated cytosine and unmethylated cytosines; performing multiplex rhPCR based enrichment on bisulfite converted nucleic acid; purification of the rhPCR amplified product of enriched nucleic acid; NGS library preparation using enriched nucleic acid; Purification of the nucleic acid libraries and performing sequencing of the enriched nucleic acid followed by data analysis. The analyzed data can be used to detect any cancer or precancerous condition in the subject.

In yet another embodiment of the invention, a universal indexing PCR can be performed for nucleic acid library preparation.

In yet another embodiment, invention also relates to a method for rhPCR based enrichment of nucleic acid to increase the sensitivity and specificity for sequencing analysis. In one example, sequence analysis of enriched nucleic acid is performed by Next generation sequencing (NGS). In another example of the invention, the sequence analysis is performed by qPCR method.

### WORKING EXAMPLES

The following examples are not intended to limit the scope of the claims to the invention, but is rather intended to be exemplary of certain embodiments. Any variations in the exemplified methods which occur to the skilled artisan are intended to fall within the scope of the present invention.

### Example 1. rhPCR Enrichment Effectivity Test with Synthetic Template

Test on spiked in synthetic template to check the enrichment and detection performance on enrichment assay followed by detection assay (Template 5000copies). The enrichment step increased methylated target 4.13˜4.82 Cts, Unmethylated target −0.95˜−1.36 Cts for Septin 9. The enrichment can increase Septin 9 target DNA by more than 10 folds.

TABLE 1
Septin 9 detection by PCR without enrichment

| Percent | | | | |
|---|---|---|---|---|
| Methylated | C_{T} Mean | | C_{T} Mean | |
| DNA in | Methylated | C_{T} | Unmethylated | C_{T} |
| unmethylated DNA | Detection Assay | SD | Detection Assay | SD |
| 0% | Undetermined | n/a | 23.80 | 0.34 |
| 0.1% | 34.45 | 0.66 | 24.00 | 0.08 |
| 1% | 31.34 | 0.08 | 24.07 | 0.05 |
| 5% | 29.03 | 0.17 | 24.33 | 0.09 |
| 10% | 27.89 | 0.10 | 24.40 | 0.32 |
| 100% | 24.46 | 0.08 | Undetermined | n/a |
| NTC | Undetermined | n/a | Undetermined | n/a |

TABLE 2
Septin 9 detection by PCR with enrichment (12 cycles)

| Percent Methylated | C_{T} Mean | | C_{T} Mean | |
|---|---|---|---|---|
| DNA in | Methylated | C_{T} | Unmethylated | C_{T} |
| unmethylated DNA | Detection Assay | SD | Detection Assay | SD |
| 0% | Undetermined | n/a | 25.16 | 0.03 |
| 0.1% | 30.13 | 0.02 | 25.28 | 0.06 |
| 1% | 26.63 | 0.03 | 25.02 | 0.15 |
| 5% | 24.44 | 0.02 | 25.31 | 0.05 |
| 10% | 23.07 | 0.11 | 25.66 | 0.04 |
| 100% | 20.32 | 0.06 | Undetermined | n/a |
| NTC | Undetermined | n/a | Undetermined | n/a |

### Example 2. rhPCR Enrichment Effectivity Test with DNA from Cancer Cell Line

Test on Separated Jurkat gDNA (9ng) and HeLa gDNA (9ng) to check the enrichment assay followed by detection assays for Septin 9. The enrichment is better for methylated target (ΔCt 9.76 for HeLa) than Unmethylated targets (ΔCt 1.96 for Jurkat). It demonstrated that the enrichment can increase Septin 9 target DNA by more than 10 folds in tumor gDNA. (Enrichment 20 cycles)

TABLE 3
Septin 9 detection by PCR with enrichment from cancer cell line DNA
Methylated Detection

| Hela DNA | | | | Jurkat DNA | | | | | |
|---|---|---|---|---|---|---|---|---|---|
| CT Mean | | CT Mean | | CT Mean | | CT Mean | | NTC | |
| Enriched | | Unenriched | | Enriched | CT | Unenriched | CT | | CT |
| Product | CT SD | Product | CT SD | Product | SD | Product | SD | Ct Mean | SD |
| 22.27 | 0.05 | 32.02 | 0.05 | Undetermined | n/a | Undetermined | n/a | Undetermined | n/a |

TABLE 4
Septin 9 detection by PCR with enrichment from cancer cell line DNA
UnMethylated Detection

| Hela DNA | | | | Jurkat DNA | | | | | |
|---|---|---|---|---|---|---|---|---|---|
| CT Mean | | CT Mean | | CT Mean | | CT Mean | | NTC | |
| Enriched | CT | Unenriched | CT | Enriched | CT | Unenriched | CT | | CT |
| Product | SD | Product | SD | Product | SD | Product | SD | Ct Mean | SD |
| Undetermined | n/a | Undetermined | n/a | 30.72 | 0.13 | 32.68 | 0.10 | Undetermined | n/a |

### Example 3. rhPCR Enrichment Effectivity Test with DNA from Colon Cancer Tissue

Test on human colon tumor gDNA (9ng) to check the enrichment assay followed by detection assay for Septin 9. The enrichment can increase Septin 9 target DNA by more than 10 folds in tumor gDNA (ΔCt 8.9 for methylated target, ΔCt 4.64 for Unmethylated target). (Enrichment 20 cycles)

TABLE 5
Septin 9 detection by PCR with enrichment from cancer tissue DNA

| Methylated Detection | | | | UnMethylated Detection | | | | | |
|---|---|---|---|---|---|---|---|---|---|
| CT Mean | | CT Mean | | CT Mean | | CT Mean | | NTC | |
| Enriched | | Unenriched | | Enriched | | Unenriched | | | CT |
| Product | CT SD | Product | CT SD | Product | CT SD | Product | CT SD | Ct Mean | SD |
| 27.26 | 0.00 | 36.15 | 0.21 | 32.18 | 0.38 | 36.81 | 0.70 | Undetermined | n/a |

### Example 4. Target Enrichment and NGS Detection

Four samples, Jurkat (25ng DNA), HeLa (25ng DNA), Spiked HeLa in Jurkat at 1% (25ng DNA) and Spiked HeLa in Jurkat at 10% (25ng DNA) were used in study. First, all DNA samples were bisulfite converted. Then did rhPCR target enrichment (14 cycles). The enrichment products were purified by SPRI method. All 4 samples were duplicated to perform universal indexing PCR (24 cycles) for NGS DNA library preparation. The 8 DNA libraries were purified again using SPRI method. A quantification PCR was performed to measure the DNA libraries quantity. Then pooled all 8 DNA libraries by equal amount. The pooled sample was loaded to Illumina MiSeq Reagent Nano Kit v2 (300 cycles, 1M clusters PF) and sequenced on MiSeq system. The NGS data was analyzed. Final read out data showed the insert mapped rate, and total C and T counted base on the CpG sites in the target region of the aim gene (Septin 9). [80] The final results indicated that there were no enrichment for unmethylated targets (Jurkat mapped rate 0.0%, 0.0%). The methylated targets were amplified specifically (HeLa mapped rate 28.9%, 39.1%). HeLa samples are almost fully methylated (Ts/Cs rate 2.31%, 2.48%). The lower amount of methylated targets were amplified during enrichment and sequenced. The sequence data also indicated full methylation in targets (1% HeLa mapped rate 25.0%, 25.9%, Ts/Cs rate 0.30%, 0.17%).

TABLE 6
Septin 9 detection by NGS with enrichment from cancer cell line DNA

| | | | | Total Cs | Total Ts | |
|---|---|---|---|---|---|---|
| | | | | in | in | |
| Sample | Total | | Pct | insert CpG | insert CpG | Ts/Cs |
| ID | Reads | Mapped | Mapped | sites | sites | Pct |
| Jurkat | 238852 | 53 | 0.0% | 0 | 0 | 0 |
| Jurkat | 264500 | 39 | 0.0% | 0 | 0 | 0 |
| HeLa | 336532 | 97164 | 28.9% | 117213 | 2705 | 2.31% |
| HeLa | 316860 | 123975 | 39.1% | 144992 | 3603 | 2.48% |
| 1% HeLa | 231772 | 57957 | 25.0% | 11224 | 34 | 0.30% |
| 1% HeLa | 244316 | 63324 | 25.9% | 13210 | 22 | 0.17% |
| 10% HeLa | 231878 | 26255 | 11.3% | 12766 | 216 | 1.69% |
| 10% HeLa | 205098 | 64950 | 31.7% | 12792 | 210 | 1.64% |

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

1. A method for detecting a methylated nucleic acid to identify a cancer or precancerous condition in a subject, the method comprising the steps of:
a. obtaining nucleic acid from a body fluid of the subject;
b. optionally, subjecting the nucleic acid to bisulfite conversion;
c. enriching the nucleic acid using rhPCR;
d. sequencing of the enriched nucleic acid using NGS; and
e. identifying a cancer or precancerous condition in the subject based on the sequencing;
wherein rhPCR increases the sensitivity and specificity for methylated nucleic acid in the NGS based sequencing analysis by selectively amplifying methylated nucleic acid and eliminating formation of primer dimers.

2. The method of claim 1, wherein the body fluid is selected from blood, plasma, serum, urine, saliva, ascites fluid, synovial fluid, amniotic fluid, semen, cerebrospinal fluid, follicular fluid and other body fluids.

3. The method of claim 1, wherein the methylated nucleic acid is selected from DNA, RNA, cDNA, mRNA, cfDNA, ccfDNA and ctDNA.

4. The method of any one of claim 1, wherein NGS sequencing is selected from Illumina's MiSeq, Illumina's HiSeq, Illumina's Genome Analyzer IIX, Roche's 454 pyrosequencing, Ion torrent semiconductor, Life Technologies's SOLiD4, Life Technologies's Ion Proton, Helicos Biosciences's Heliscope and Pacific Biosciences's SMRT.

5. The method of claim 1, wherein the cancer or precancerous condition is colon cancer, liver cancer, brain cancer, uterine cancer, bladder cancer, blood cancer, lung adenocarcinomas, breast cancer, thyroid carcinoma, pancreatic cancer, papillary thyroid carcinoma, ovarian carcinoma, gastric carcinoma, malignant, mesothelioma, prostate carcinoma, neuroblastic tumors, colorectal carcinoma, spitzoid, melanoma, salivary, adenoid, cystic, carcinoma, multiforme, stomach cancer, kidney cancer, urethral cancer, glioblastoma, oral squamous cell, carcinoma, mastocytosis, extramammary Paget's disease, Acute Myeloid, Leukemia, cholangiocarcinomaor sarcoma.

6. A method for detecting a methylated nucleic acid to identify a cancer or precancerous condition in a subject, the method comprising the steps of:
a. obtaining nucleic acid from body fluid of the subject;
b. optionally, subjecting the nucleic acid to bisulfite conversion;
c. enriching the nucleic acid using rhPCR;
d. sequencing of the enriched nucleic acid using qPCR or RT-PCR (Real-time PCR); and
e. identifying a cancer or precancerous condition in the subject based on the sequencing;
wherein rhPCR increases the sensitivity and specificity for methylated nucleic acid in the qPCR or RT-PCR (Real-time PCR) based sequencing analysis by selectively amplifying methylated nucleic acid and eliminating formation of primer dimers.

7. The method of claim 6, wherein the body fluid is selected from blood, plasma, serum, urine, saliva, ascites fluid, synovial fluid, amniotic fluid, semen, cerebrospinal fluid, follicular fluid and other body fluids.

8. The method of claim 6, wherein the methylated nucleic acid is selected from DNA, RNA, cDNA, mRNA, cfDNA, ccfDNA and ctDNA.

9. The method of claim 6, wherein the cancer or precancerous condition is colon cancer, liver cancer, brain cancer, uterine cancer, bladder cancer, blood cancer, lung adenocarcinomas, breast cancer, thyroid carcinoma, pancreatic cancer, papillary thyroid carcinoma, ovarian carcinoma, gastric carcinoma, malignant, mesothelioma, prostate carcinoma, neuroblastic tumors, colorectal carcinoma, spitzoid, melanoma, salivary, esophageal carcinoma, adenoid, cystic, carcinoma, multiforme, stomach cancer, kidney cancer, urethral cancer, glioblastoma, oral squamous cell, carcinoma, mastocytosis, extramammary Paget's disease, Acute Myeloid, Leukemia, cholangiocarcinomaor sarcoma.

10. A method for increasing sensitivity and specificity for methylated DNA detection using NGS based sequencing analysis and rhPCR, where rhPCR is used to enrich the methylated DNA.

11. The method of claim 10, wherein the methylated DNA is selected from cDNA, cfDNA, ccfDNA and ctDNA.

12. The method of claim 10, wherein the methylated DNA detection is further used to identify a cancer or precancerous condition in a subject.

13. The method of claim 12, wherein the cancer or precancerous condition is colorectal cancer.

14. The method of claim 10, wherein NGS sequencing is selected from Illumina's MiSeq, Illumina's HiSeq, Illumina's Genome Analyzer IIX, Roche's 454 pyrosequencing, Ion torrent semiconductor, Life Technologies's SOLiD4, Life Technologies's Ion Proton, Helicos Biosciences's Heliscope and Pacific Biosciences's SMRT.

15. The method of claim 10, wherein the rhPCR is preceded by bisulfite conversion.

16.-20. (canceled)
